(19) United States
(12) Patent Application Publication
(10) Pub. No.: US 2025/0259380 A1
(43) Pub. Date: Aug. 14, 2025

(54) SYSTEM FOR OPTIMIZING SENSOR SETTINGS IN A MULTI-CAMERA ENVIRONMENT BASED ON FOUNDATION MODELS AND HISTORICAL DATA

(71) Applicant: MICROSOFT TECHNOLOGY LICENSING, LLC, Redmond, WA (US)

(72) Inventors: Andréa BRITTO MATTOS LIMA, Sao Paulo (BR); Christopher Patrick O'DOWD, Seattle, WA (US); Spencer G. FOWERS, Duvall, WA (US); Thiago VALLIN SPINA, Campinas (BR)

(21) Appl. No.: 18/440,643

(22) Filed: Feb. 13, 2024

(51) Int. Cl. G06T 17/00; G16H 50/20; G16H 80/00; H04N 23/11

(52) U.S. Cl. CPC G06T 17/00; G16H 50/20; G16H 80/00; H04N 23/11

(57) ABSTRACT

3D teleconferences use an array of cameras to generate a 3D model of a subject. During a calibration and registration process the pose of each camera may be adjusted. Similarly, camera settings such as focus depth and white balance may be modified. These changes are made to improve the quality of the 3D model generated from image data captured by the cameras. Many factors affect the quality of images captured by the cameras. For example, depth sensors may be affected by the skin tone of the subject. In some configurations, a machine learning model (ML model) is trained on adjustments to properties that affect 3D model quality. The resulting ML model may then be used to infer camera adjustments for a given set of subject attributes, camera properties, and/or environment properties.

### BACKGROUND

In an increasingly digital world, where remote work and global collaboration have become the norm, high fidelity 3D video conferencing represents a transformative leap in communication technology. This advanced form of conferencing transcends the limitations of traditional 2D video calls, providing a more immersive and engaging experience that closely replicates face-to-face interactions. High fidelity 3D conferencing facilitates better comprehension of non-verbal cues, such as body language and spatial awareness, enhancing the overall quality and effectiveness of communication. In the healthcare context, real-time 3D interaction with patients enables remote practitioners to better diagnose and treat disease.

It is with respect to these and other considerations that the disclosure made herein is presented.

### SUMMARY

3D teleconferences use an array of cameras to generate a 3D model of a subject. During a calibration and registration process the pose of each camera may be adjusted. Similarly, camera settings such as focus depth and white balance may be modified. These changes are made to improve the quality of the 3D model generated from image data captured by the cameras. Many factors affect the quality of images captured by the cameras. For example, depth sensors may be affected by the skin tone of the subject. In some configurations, a machine learning model (ML model) is trained on adjustments to properties that affect 3D model quality. The resulting ML model may then be used to infer camera adjustments for a given set of subject attributes, camera properties, and/or environment properties.

Features and technical benefits other than those explicitly described above will be apparent from a reading of the following Detailed Description and a review of the associated drawings. This Summary is provided to introduce a selection of concepts in a simplified form that are further described below in the Detailed Description. This Summary is not intended to identify key or essential features of the claimed subject matter, nor is it intended to be used as an aid in determining the scope of the claimed subject matter. The term “techniques,” for instance, may refer to system(s), method(s), computer-readable instructions, module(s), algorithms, hardware logic, and/or operation(s) as permitted by the context described above and throughout the document.

### BRIEF DESCRIPTION OF THE DRAWINGS

The Detailed Description is described with reference to the accompanying figures. In the figures, the left-most digit(s) of a reference number identifies the figure in which the reference number first appears. The same reference numbers in different figures indicate similar or identical items. References made to individual items of a plurality of items can use a reference number with a letter of a sequence of letters to refer to each individual item. Generic references to the items may use the specific reference number without the sequence of letters.

FIG. 1 illustrates multiple depth cameras capturing images of a subject.

FIG. 2 illustrates properties that affect the quality of a 3D model generated from image streams captured by multiple depth cameras.

FIG. 3 illustrates training a camera property ML model.

FIG. 4 illustrates the ML model inferring camera properties from a collection of subject attributes, camera properties, and environment properties.

FIG. 5 is a flow diagram of an example method for optimizing sensor settings in a multi-camera environment.

FIG. 6 is a computer architecture diagram illustrating an illustrative computer hardware and software architecture for a computing system capable of implementing aspects of the techniques and technologies presented herein.

### DETAILED DESCRIPTION

Disclosed are techniques for automatically adjusting camera pose and other settings in a 3D modeling environment. In some configurations, a machine learning model is used to generate settings for cameras of an array of cameras. Images generated by the array of cameras are used to construct a 3D model, such as with MICROSOFT HOLOPORTATION technology. Properly configuring settings of the cameras results in improved fidelity of the 3D model.

In some configurations, manual adjustments made to camera settings are used to train the machine learning model. For example, a mapping from subject attributes, initial camera settings, and environmental settings to adjusted camera settings is created as a user manually refines camera settings. This mapping is used to train the ML model to infer updated camera settings.

For example, a camera array may be used to capture images of a medical patient. A practitioner, sitting remotely, views a 3D model representation of the patient. The 3D model is generated from image streams received from the camera array. Each camera in the array has initial camera settings, while the patient has a set of attributes, such as skin tone. The practitioner notices that, due to the patient's light skin tone, depth perception is inaccurate, resulting in a distorted representation of the patient. The practitioner adjusts the infra-red settings of the cameras until depth perception improves. The ML model is trained on a mapping from the initial settings and patient attributes to the settings selected by the practitioner.

When a new consultation starts, the ML model suggests optimal camera poses and other settings given the new patient's attributes and a new set of initial camera settings. A clinician in the room with the patient may move the cameras accordingly or use a mechanical apparatus to have the cameras moved to their optimal locations.

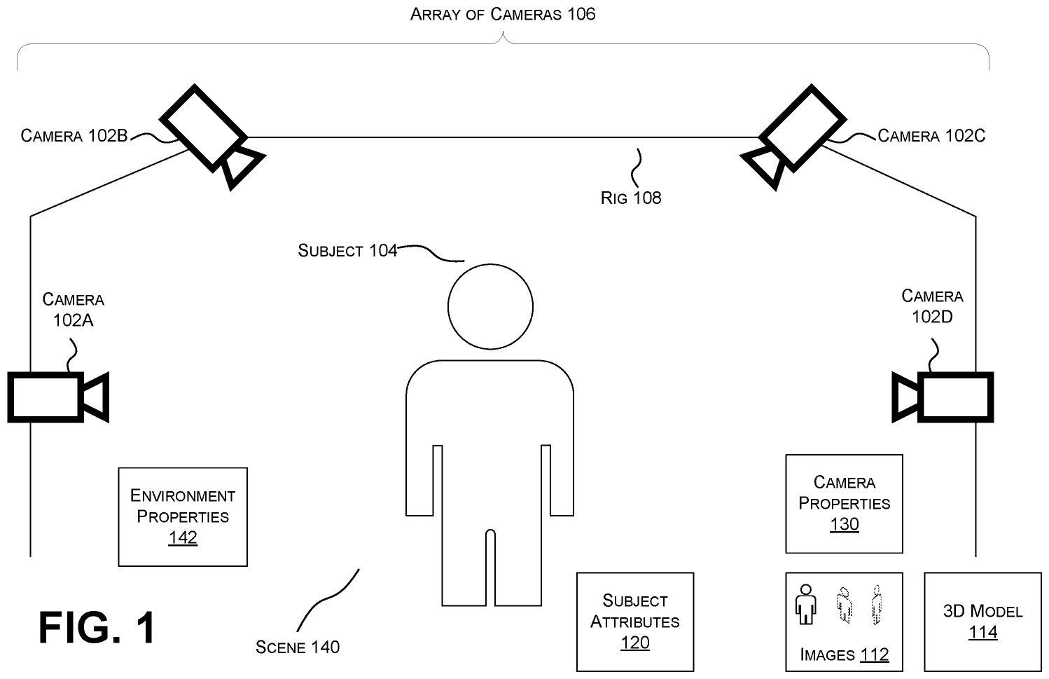

FIG. 1 illustrates multiple cameras 102 capturing images 112 of subject 104. Cameras 102 may be attached to rig 108, which holds cameras 102 in place so as to view scene 140 from different perspectives. Cameras 102, which are collectively referred to as array of cameras 106, capture color images of scene 140 as well as depth maps that precisely measure a distance from the camera for each pixel of an image. For example, camera 102A may include a synchronized RGB camera for capturing color and a depth camera for capturing depth.

Cameras 102 are configured according to camera properties 130. The pose of each camera 102—the location and orientation of each camera—is one example of a camera property 130. Placement of a camera on rig 108 may determine the location and/or the pose of that camera. The pose of a camera 102 may be manually or automatically adjusted. Other camera properties 130 that affect the quality of the generated 3D model includes white balance, brightness, resolution, focal length, color saturation, and the like. Some or all of camera properties 130 may be adjusted to improve the quality of images 112 and/or the 3D model 114 generated from images 112.

Other factors affect the quality of images 112 and the resulting 3D model 114, such as subject attributes 120 and environment properties 142. Subject attributes 120 include physical attributes of subject 104, a medical history, and any other information that is particular to subject 104 that could affect the quality of images 112 or the resulting 3D model 114. Environmental properties 142 may include room dimensions, the extent of rig 108, and other limitations on camera placement, as well as ambient brightness. In some configurations, subject attributes 120 and/or environment properties 142 are manually entered, while in other configurations subject attributes 120 and/or environment properties 142 are inferred from images 112.

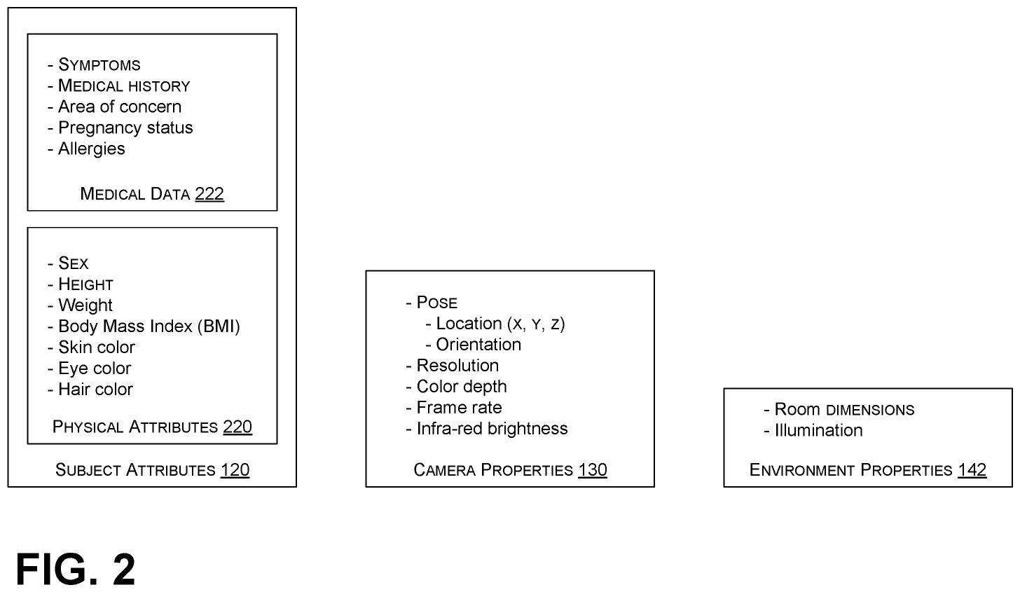

FIG. 2 illustrates properties that affect the quality of 3D model 114 generated from image streams 112 captured by cameras 102. Subject attributes 120 may include physical attributes 220 and medical data 222. Physical attributes 220 refer to attributes about subject 104. When subject 104 is a person, attributes 220 such as the person's sex, height, weight, body mass index, skin color, eye color, and hair color may be noted. This information may be entered manually or determined programmatically by an image analysis module. However, people are only one example of subject 104—subject 104 may be an animal, a product, an object in need of repair, a demonstration, or the like.

In some contexts, 3D model 114 of subject 104 is created for use in a telemedicine scenario. A doctor or other practitioner may remotely view 3D model 114 of subject 104 in order to evaluate subject 104 for a procedure, or as part of follow-up care. In these contexts, medical data 222 may be useful to know when configuring cameras 102. Medical data 222 may include any information that could affect images 112 or the resulting 3D model 114, such as symptoms subject 104 is having, the medical history of subject 104, and area of concern, such as a particular body part, pregnancy status, allergies, or any other condition that may affect how subject 104 is perceived by cameras 102.

As discussed briefly above, camera properties 130 may include a pose of camera 102, which includes a location in (x, y, z) coordinates as well as a camera orientation. Camera orientation may be defined in terms of spherical coordinates. Other camera properties 130 include camera resolution, color depth, and frame rate. Some depth cameras use infra-red to measure a per-pixel distance. In these scenarios, infra-red brightness is a setting that may be adjusted to account for differences in subjects 104.

Environment properties 142, such as room dimensions and illumination levels, add additional information for use with a machine learning model. Ambient illumination levels have a significant effect on the clarity color quality of images 112. Room dimensions constrain where cameras 102 may be placed.

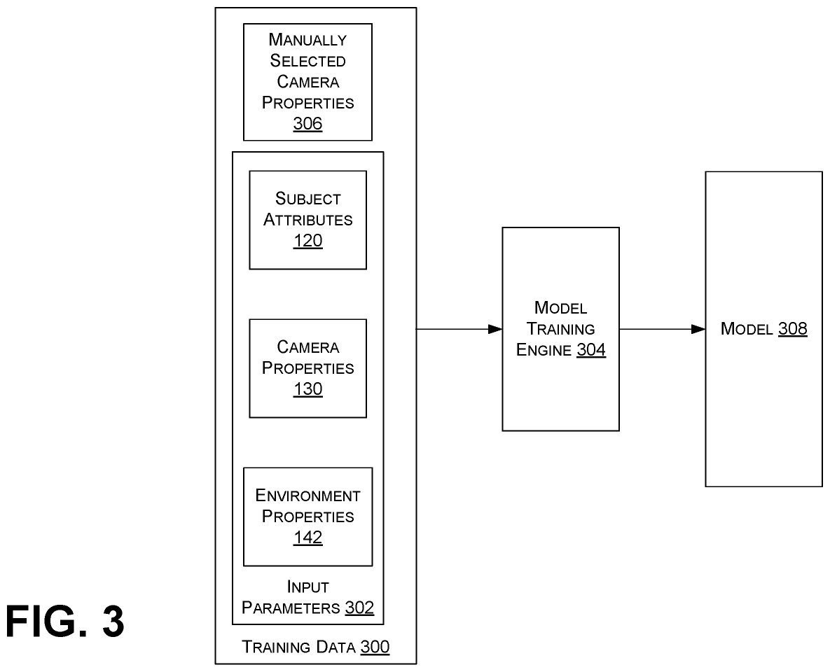

FIG. 3 illustrates training a camera property ML model 308. Specifically, model training engine 304 applies training data 300 to train camera property ML model 308—also referred to as ML model 308. Training data 300 includes a mapping from input parameters 302 to manually selected camera properties 306. Input parameters 302 may include subject attributes 120, camera properties 130, and environment properties 142, as discussed above. Input parameters 302 may represent initial attributes and properties of scene 140 and cameras 102. When training model 308, these input parameters are mapped to manually selected camera properties 306. Manually selected camera properties 306 represent the camera properties that an operator manually settled on for creating the highest quality 3D model. In this way, manually selected camera properties 306 are the ‘ground truth’ with which to train model 308. In this way, model 308 learns what camera properties, such as pose, white balance, brightness, etc., will yield the best 3D model.

Manually selected camera properties 306 may include some or all of the same properties included in camera properties 130. Manually selected camera properties 306 may also include additional properties for configuring cameras 102 that are not included in camera properties 130.

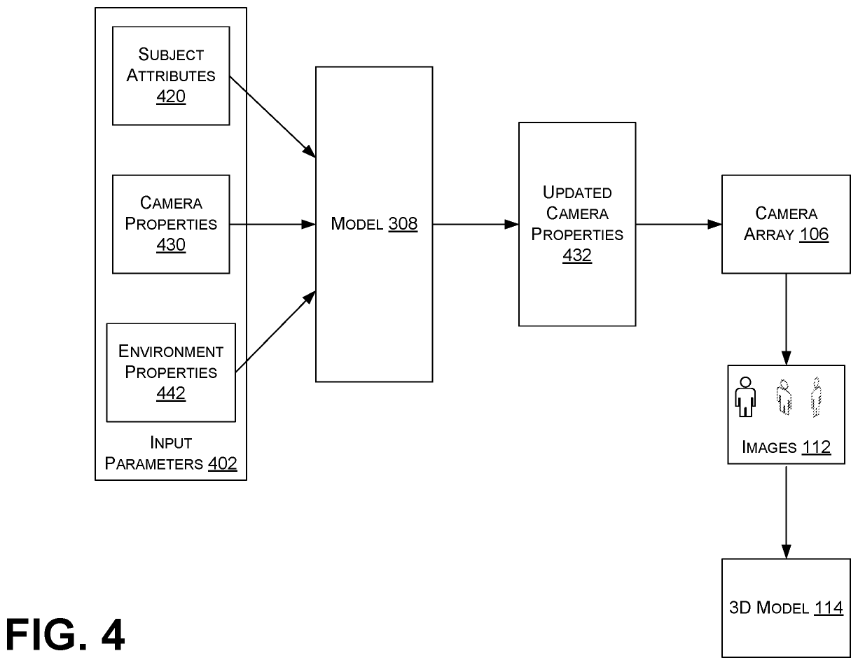

FIG. 4 illustrates ML model 308 inferring updated camera properties 432 from a collection of subject attributes, camera properties, and environment properties. For example, input parameters 402 include subject attributes 420, camera properties 430, and environment properties 442. These attributes and properties may be manually entered or programmatically determined from images 112 of scene 140. Input parameters 402 are provided to model 308 and used to infer updated camera properties 432. Similar to manually selected camera properties 306, updated camera properties 432 may include the same types of properties as camera properties 130—pose, white balance, focal length, etc.

In some configurations, updated camera properties 432 may be used to automatically configure camera array 106. Additionally, or alternatively, updated camera properties 432 may be displayed to a user, such as a practitioner in a telemedicine session, as suggestions that may be manually adopted. Re-configuring camera array 106 may cause individual cameras to change location or orientation. Other settings, such as white balance, brightness, color saturation, and the like, may also be set.

Once camera array 106 has been re-configured with updated camera properties 432, images 112 streamed from cameras 102 may be used to synthesize 3D model 114. 3D model 114 may be displayed in a holographic projector, holographic classes, virtual reality headsets, computer monitor, or other device capable of displaying a 3D rendering of subject 104.

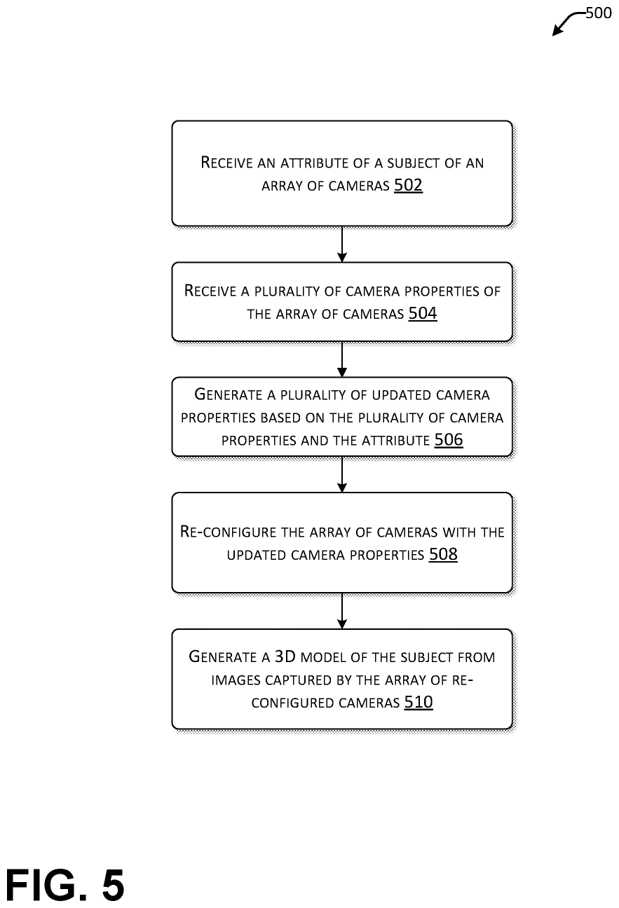

With reference to FIG. 5, routine 500 begins at operation 502, where an attribute 120 of a subject 104 of an array of cameras 106 is received. One common scenario is remote medical assistance, such as evaluating subject 104 for surgery, performing a follow-up visit, or the like. Patients such as subject 104 may be evaluated for conditions such as tumors, burns, trauma, etc. For example, a person with a tumor may be evaluated remotely for possible reconstructive surgery. The patient may be evaluated for the location of donor tissue, such as a skin graft or blood vessels. These types of evaluations are made visually, and so there is a significant advantage to enabling this type of analysis over a 3D teleconference as compared to a 2D teleconference.

Next at operation 504, a plurality of camera properties 130 of array of cameras 106 is received. One example camera property is the pose of each camera 102. Relative poses of cameras 102 may be estimated during a calibration and registration process. For example, the poses of each camera 102 may be estimated by holding up a board of known dimensions within the field of view of each camera 102. The board may have a graphical pattern with known geometry that allows a calibration algorithm to compute the extrinsic and intrinsic properties of the cameras using multi-view geometry. Relative poses of cameras 102 may then be estimated based on the size and orientation of the board in images taken by each camera 102. Calibration and registration may also include estimation of intrinsic properties 130 of each camera 102, such as focal length, principal point, and distortion.

Some settings 130 of cameras 102 can affect the color representation of generated 3D model 114. For example, the exposure setting 130 can affect brightness while white balance can affect color temperatures, both of which affect the color of 3D model 114. Other settings 130 can affect the level of detail and scene coverage. For example, resolution and field of view can affect the level of detail and the coverage of generated 3D model 114.

For example, if a patient with a tumor is being evaluated by a practitioner, the practitioner may need to adjust the RGB camera settings such as exposure, contrast, brightness, and white balance to highlight the tumor and capture detailed information about its shape and location, as well as an accurate color representation. If the patient has a darker skin tone, the practitioner may need to increase the infrared illumination of the depth sensor to compensate for higher absorption of infrared light by darker skin tones. The practitioner may also adjust the exposure settings of the RGB cameras to ensure that the skin tone is accurately represented. If the area of interest is large, the practitioner may need to adjust the position and/or field of view of the cameras to ensure complete coverage of the area. If the patient is expected to move during the session, the practitioner may need to adjust the capture time and settings of the cameras to ensure that the captured images are not blurry due to the patient's movements.

Next at operation 506, a plurality of updated camera properties 432 are generated based on the plurality of camera properties 130 and the attribute 120.

Next at operation 508, the array of cameras 106 are reconfigured using the updated camera properties 432.

Next at operation 510, a 3D model 114 of subject 104 is generated from images 112 captured by the array of re-configured cameras 106. In the medical context, a practitioner may rotate 3D model 114 in order to obtain different perspectives of the patient. The practitioner may similarly zoom in and out. The practitioner may leave notes on specific body parts of the patient, such as the location of potential donor tissue. On subsequent visits by subject 104, notes left on specific body parts may be displayed or made available on the corresponding body parts in the 3D model.

The particular implementation of the technologies disclosed herein is a matter of choice dependent on the performance and other requirements of a computing device. Accordingly, the logical operations described herein are referred to variously as states, operations, structural devices, acts, or modules. These states, operations, structural devices, acts, and modules can be implemented in hardware, software, firmware, in special-purpose digital logic, and any combination thereof. It should be appreciated that more or fewer operations can be performed than shown in the figures and described herein. These operations can also be performed in a different order than those described herein.

It also should be understood that the illustrated methods can end at any time and need not be performed in their entireties. Some or all operations of the methods, and/or substantially equivalent operations, can be performed by execution of computer-readable instructions included on a computer-storage media, as defined below. The term “computer-readable instructions,” and variants thereof, as used in the description and claims, is used expansively herein to include routines, applications, application modules, program modules, programs, components, data structures, algorithms, and the like. Computer-readable instructions can be implemented on various system configurations, including single-processor or multiprocessor systems, minicomputers, mainframe computers, personal computers, hand-held computing devices, microprocessor-based, programmable consumer electronics, combinations thereof, and the like.

Thus, it should be appreciated that the logical operations described herein are implemented (1) as a sequence of computer implemented acts or program modules running on a computing system and/or (2) as interconnected machine logic circuits or circuit modules within the computing system. The implementation is a matter of choice dependent on the performance and other requirements of the computing system. Accordingly, the logical operations described herein are referred to variously as states, operations, structural devices, acts, or modules. These operations, structural devices, acts, and modules may be implemented in software, in firmware, in special purpose digital logic, and any combination thereof.

For example, the operations of the routine 500 are described herein as being implemented, at least in part, by modules running the features disclosed herein can be a dynamically linked library (DLL), a statically linked library, functionality produced by an application programing interface (API), a compiled program, an interpreted program, a script or any other executable set of instructions. Data can be stored in a data structure in one or more memory components. Data can be retrieved from the data structure by addressing links or references to the data structure.

Although the following illustration refers to the components of the figures, it should be appreciated that the operations of the routines 500 may be also implemented in many other ways. For example, the routine 500 may be implemented, at least in part, by a processor of another remote computer or a local circuit. In addition, one or more of the operations of the routine 500 may alternatively or additionally be implemented, at least in part, by a chipset working alone or in conjunction with other software modules. In the example described below, one or more modules of a computing system can receive and/or process the data disclosed herein. Any service, circuit or application suitable for providing the techniques disclosed herein can be used in operations described herein.

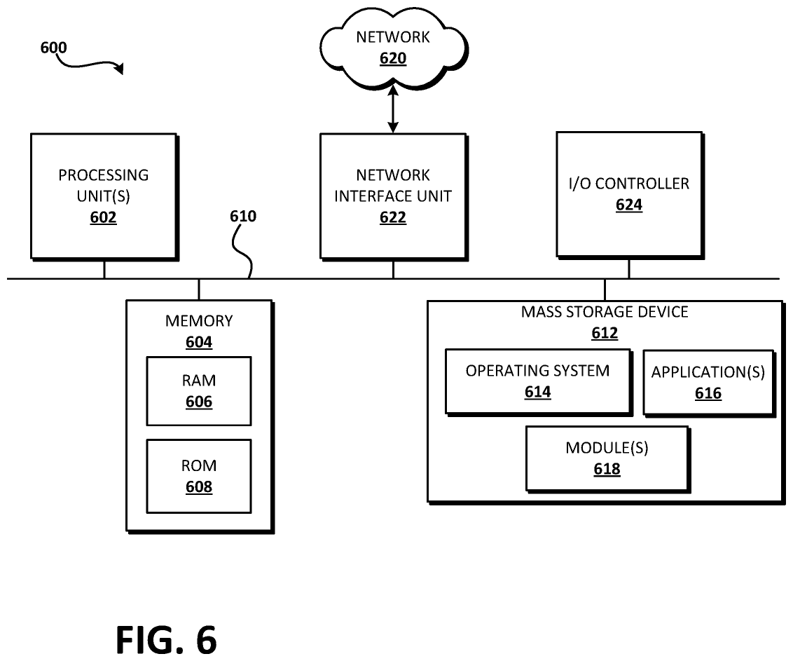

FIG. 6 shows additional details of an example computer architecture 600 for a device, such as a computer or a server configured as part of the systems described herein, capable of executing computer instructions (e.g., a module or a program component described herein). The computer architecture 600 illustrated in FIG. 6 includes processing unit(s) 602, a system memory 604, including a random-access memory 606 (“RAM”) and a read-only memory (“ROM”) 608, and a system bus 610 that couples the memory 604 to the processing unit(s) 602.

Processing unit(s), such as processing unit(s) 602, can represent, for example, a CPU-type processing unit, a GPU-type processing unit, a neural processing unit, a field-programmable gate array (FPGA), another class of digital signal processor (DSP), or other hardware logic components that may, in some instances, be driven by a CPU. For example, and without limitation, illustrative types of hardware logic components that can be used include Application-Specific Integrated Circuits (ASICs), Application-Specific Standard Products (ASSPs), System-on-a-Chip Systems (SOCs), Complex Programmable Logic Devices (CPLDs), Neural Processing Unites (NPUs) etc.

A basic input/output system containing the basic routines that help to transfer information between elements within the computer architecture 600, such as during startup, is stored in the ROM 608. The computer architecture 600 further includes a mass storage device 612 for storing an operating system 614, application(s) 616, modules 618, and other data described herein.

The mass storage device 612 is connected to processing unit(s) 602 through a mass storage controller connected to the bus 610. The mass storage device 612 and its associated computer-readable media provide non-volatile storage for the computer architecture 600. Although the description of computer-readable media contained herein refers to a mass storage device, it should be appreciated by those skilled in the art that computer-readable media can be any available computer-readable storage media or communication media that can be accessed by the computer architecture 600.

Computer-readable media can include computer-readable storage media and/or communication media. Computer-readable storage media can include one or more of volatile memory, nonvolatile memory, and/or other persistent and/or auxiliary computer storage media, removable and non-removable computer storage media implemented in any method or technology for storage of information such as computer-readable instructions, data structures, program modules, or other data. Thus, computer storage media includes tangible and/or physical forms of media included in a device and/or hardware component that is part of a device or external to a device, including but not limited to random access memory (RAM), static random-access memory (SRAM), dynamic random-access memory (DRAM), phase change memory (PCM), read-only memory (ROM), erasable programmable read-only memory (EPROM), electrically erasable programmable read-only memory (EEPROM), flash memory, compact disc read-only memory (CD-ROM), digital versatile disks (DVDs), optical cards or other optical storage media, magnetic cassettes, magnetic tape, magnetic disk storage, magnetic cards or other magnetic storage devices or media, solid-state memory devices, storage arrays, network attached storage, storage area networks, hosted computer storage or any other storage memory, storage device, and/or storage medium that can be used to store and maintain information for access by a computing device.

In contrast to computer-readable storage media, communication media can embody computer-readable instructions, data structures, program modules, or other data in a modulated data signal, such as a carrier wave, or other transmission mechanism. As defined herein, computer storage media does not include communication media. That is, computer-readable storage media does not include communications media consisting solely of a modulated data signal, a carrier wave, or a propagated signal, per se.

According to various configurations, the computer architecture 600 may operate in a networked environment using logical connections to remote computers through the network 620. The computer architecture 600 may connect to the network 620 through a network interface unit 622 connected to the bus 610. The computer architecture 600 also may include an input/output controller 624 for receiving and processing input from a number of other devices, including a keyboard, mouse, touch, or electronic stylus or pen. Similarly, the input/output controller 624 may provide output to a display screen, a printer, or other type of output device.

It should be appreciated that the software components described herein may, when loaded into the processing unit(s) 602 and executed, transform the processing unit(s) 602 and the overall computer architecture 600 from a general-purpose computing system into a special-purpose computing system customized to facilitate the functionality presented herein. The processing unit(s) 602 may be constructed from any number of transistors or other discrete circuit elements, which may individually or collectively assume any number of states. More specifically, the processing unit(s) 602 may operate as a finite-state machine, in response to executable instructions contained within the software modules disclosed herein. These computer-executable instructions may transform the processing unit(s) 602 by specifying how the processing unit(s) 602 transition between states, thereby transforming the transistors or other discrete hardware elements constituting the processing unit(s) 602.

The present disclosure is supplemented by the following example clauses:

Example 1: A method comprising: receiving an attribute of a subject of an array of cameras; receiving a plurality of camera properties of the array of cameras; generating a plurality of updated camera properties based on the plurality of camera properties and the attribute; re-configuring the array of cameras with the updated camera properties; and generating a 3D model of the subject from images captured by the array of re-configured cameras.

Example 2: The method of example 1, wherein the subject comprises a medical patient, and wherein the attribute of the subject comprises a medical diagnosis of the medical patient.

Example 3: The method of example 2, wherein the medical diagnosis is inferred from an initial set of images obtained from the array of cameras.

Example 4: The method of example 1, wherein the updated camera properties comprise updated camera poses for the array of cameras.

Example 5: The method of example 1, wherein the camera properties comprise camera poses for the array of cameras.

Example 6: The method of example 1, wherein the camera properties comprise white balance or brightness.

Example 7: The method of example 1, wherein the camera properties comprise resolution or field of view.

Example 8: The method of example 1, wherein the updated camera properties are inferred from a machine learning model, wherein the camera properties are inputs to the machine learning model, and wherein the updated camera properties are generated by the machine learning model.

Example 9: A system comprising: a processing unit; and a computer-readable storage medium having computer-executable instructions stored thereupon, which, when executed by the processing unit, cause the processing unit to: receive an attribute of a subject of an array of cameras; receive a plurality of camera properties of the array of cameras; receive a plurality of updated camera properties of the array of cameras, wherein the plurality of updated camera properties are set in response to viewing a 3D model of the subject, wherein the 3D model is generated from images taken by the array of cameras; and train a camera property machine learning model with training data comprising a mapping of the attribute of the subject and the plurality of camera properties to the updated camera properties.

Example 10: The system of example 9, wherein the plurality of updated camera properties comprises updated camera poses of the array of cameras.

Example 11: The system of example 9, wherein the array of cameras comprises an array of depth cameras.

Example 12: The system of example 11, wherein the user comprises a medical practitioner, and wherein the updated camera properties adjust an infra-red illumination level of a depth sensor of the array of depth cameras.

Example 13: The system of example 9, wherein the training data comprises an environment boundary.

Example 14: The system of example 9, wherein the attribute of the subject comprises medical history data of the subject.

Example 15: The system of example 9, wherein the attribute of the subject comprises physical characteristics about the subject.

Example 16: A computer-readable storage medium having encoded thereon computer-readable instructions that when executed by a processing unit causes a system to: receive an attribute of a subject of an array of cameras; receive a plurality of camera properties of the array of cameras; generate a plurality of updated camera properties based on the plurality of camera properties and the attribute; re-configure the array of cameras with the updated camera properties; and generate a 3D model of the subject from images captured by the array of re-configured cameras.

Example 17: The computer-readable storage medium of example 16, wherein the attribute of the subject comprises a body part of the subject.

Example 18: The computer-readable storage medium of example 16, wherein the array of cameras are re-configured in real time in response to changes to the attribute of the subject.

Example 19: The computer-readable storage medium of example 16, wherein the subject comprises an object being inspected for sale or for repair.

Example 20: The computer-readable storage medium of example 16, wherein the plurality of updated camera properties are inferred from a camera property machine learning model trained on a mapping of individual camera properties to individual updated camera properties.

While certain example embodiments have been described, these embodiments have been presented by way of example only and are not intended to limit the scope of the inventions disclosed herein. Thus, nothing in the foregoing description is intended to imply that any particular feature, characteristic, step, module, or block is necessary or indispensable. Indeed, the novel methods and systems described herein may be embodied in a variety of other forms; furthermore, various omissions, substitutions and changes in the form of the methods and systems described herein may be made without departing from the spirit of the inventions disclosed herein. The accompanying claims and their equivalents are intended to cover such forms or modifications as would fall within the scope and spirit of certain of the inventions disclosed herein.

It should be appreciated that any reference to “first,” “second,” etc. elements within the Summary and/or Detailed Description is not intended to and should not be construed to necessarily correspond to any reference of “first,” “second,” etc. elements of the claims. Rather, any use of “first” and “second” within the Summary, Detailed Description, and/or claims may be used to distinguish between two different instances of the same element.

In closing, although the various techniques have been described in language specific to structural features and/or methodological acts, it is to be understood that the subject matter defined in the appended representations is not necessarily limited to the specific features or acts described. Rather, the specific features and acts are disclosed as example forms of implementing the claimed subject matter.

What is claimed is:

1. A method comprising:
receiving an attribute of a subject of an array of cameras;
receiving a plurality of camera properties of the array of cameras;
generating a plurality of updated camera properties based on the plurality of camera properties and the attribute;
re-configuring the array of cameras with the updated camera properties; and
generating a 3D model of the subject from images captured by the array of re-configured cameras.

2. The method of claim 1, wherein the subject comprises a medical patient, and wherein the attribute of the subject comprises a medical diagnosis of the medical patient.

3. The method of claim 2, wherein the medical diagnosis is inferred from an initial set of images obtained from the array of cameras.

4. The method of claim 1, wherein the updated camera properties comprise updated camera poses for the array of cameras.

5. The method of claim 1, wherein the camera properties comprise camera poses for the array of cameras.

6. The method of claim 1, wherein the camera properties comprise white balance or brightness.

7. The method of claim 1, wherein the camera properties comprise resolution or field of view.

8. The method of claim 1, wherein the updated camera properties are inferred from a machine learning model, wherein the camera properties are inputs to the machine learning model, and wherein the updated camera properties are generated by the machine learning model.

9. A system comprising:
a processing unit; and
a computer-readable storage medium having computer-executable instructions stored thereupon, which, when executed by the processing unit, cause the processing unit to:
receive an attribute of a subject of an array of cameras;
receive a plurality of camera properties of the array of cameras;
receive a plurality of updated camera properties of the array of cameras, wherein the plurality of updated camera properties are set in response to viewing a 3D model of the subject, wherein the 3D model is generated from images taken by the array of cameras; and
train a camera property machine learning model with training data comprising a mapping of the attribute of the subject and the plurality of camera properties to the updated camera properties.

10. The system of claim 9, wherein the plurality of updated camera properties comprises updated camera poses of the array of cameras.

11. The system of claim 9, wherein the array of cameras comprises an array of depth cameras.

12. The system of claim 11, wherein the user comprises a medical practitioner, and wherein the updated camera properties adjust an infra-red illumination level of a depth sensor of the array of depth cameras.

13. The system of claim 9, wherein the training data comprises an environment boundary.

14. The system of claim 9, wherein the attribute of the subject comprises medical history data of the subject.

15. The system of claim 9, wherein the attribute of the subject comprises physical characteristics about the subject.

16. A computer-readable storage medium having encoded thereon computer-readable instructions that when executed by a processing unit causes a system to:
receive an attribute of a subject of an array of cameras;
receive a plurality of camera properties of the array of cameras;
generate a plurality of updated camera properties based on the plurality of camera properties and the attribute;
re-configure the array of cameras with the updated camera properties; and
generate a 3D model of the subject from images captured by the array of re-configured cameras.

17. The computer-readable storage medium of claim 16, wherein the attribute of the subject comprises a body part of the subject.

18. The computer-readable storage medium of claim 16, wherein the array of cameras are re-configured in real time in response to changes to the attribute of the subject.

19. The computer-readable storage medium of claim 16, wherein the subject comprises an object being inspected for sale or for repair.

20. The computer-readable storage medium of claim 16, wherein the plurality of updated camera properties are inferred from a camera property machine learning model trained on a mapping of individual camera properties to individual updated camera properties.
